(19) United States
(12) Patent Application Publication
(10) Pub. No.: US 2009/0177186 A1
(43) Pub. Date: Jul. 9, 2009

(54) Luer Lock Connector with Roughened Tip

(76) Inventor: Peter Delano, Oakland, NJ (US)

Correspondence Address: David W. Highet, VP & Chief IP Counsel;Becton, Dickinson and Company, (Diehl Servilla, LLC), 1 Becton Drive, MC 110, Franklin Lakes, NJ, 07417-1880, US

(21) Appl. No.: 11/970,834

(22) Filed: Jan. 8, 2008

(51) Int. Cl. A61M 39/10; B21D 39/00

(52) U.S. Cl. 604/534; 29/428

(57) ABSTRACT

Medical devices with male luer lock fittings with either a male conical tip having a roughened outer surface or a female luer lock fitting with a roughened surface and methods for manufacture and use are disclosed. The outer surface of the male conical tip or a surface of the female luer lock fitting is roughened to decrease the likelihood of breakage during disassembly from a female luer lock fitting of another medical device in the presence of a liquid.

### FIELD OF THE INVENTION

The present invention relates in general to the connector portion of a medical device. More particularly, embodiments of the disclosed invention are directed to the surface finish of a luer lock fitting having a conical tip. Methods of manufacturing a male luer lock fitting device with a rough surface are also disclosed.

### BACKGROUND OF THE INVENTION

Medical devices having male luer lock fittings, for example, male luer lock syringes, are often assembled with female tube adapters or tube extensions. As described in International Standard ISO 594-2, published by the International Organization for Standardization, such devices include a male conical fitting in a coaxial relation with an internally threaded collar. Female luer lock fittings having external lugs for engaging the internally threaded collar are assembled to the male conical tip by applying a torque to the female luer lock fitting until the female fitting is sufficiently tight.

It has been observed that the male conical tip of a male luer lock fitting can fail and break off during application of torque to disassemble the male luer lock fitting from a female luer lock fitting. This is particularly true when the male luer locking fitting and female luer lock fitting have been assembled in the presence of a liquid, and the fittings have been assembled for a certain period of time. When this occurs, the male conical tip of the male luer lock fitting breaks off at the base. This leaves the conical tip lodged in the female luer lock fitting rendering it useless, necessitating replacement.

### SUMMARY OF THE INVENTION

One or more embodiments of the invention are directed toward medical devices comprising an adapter portion for connection to a fitting. The adapter portion includes a male luer lock fitting having an internally threaded collar in a coaxial relationship with a male conical tip having an outside surface that mates upon application of torque with a female luer lock fitting. The female luer lock fitting has lugs that engage the threaded collar of the male luer lock fitting. Either one or both of the outside surfaces of the male conical tip or a surface of the female luer lock fitting is roughened such that after assembly of the male luer lock fitting with the female luer lock fitting while liquid is present between the male conical tip and the female luer lock fitting, the torque required to remove the male luer lock fitting from the female luer fitting after being assembled 24 hours is less than the torque required to remove a conventional male luer lock fitting from a female luer lock fitting assembled under similar conditions after being assembled for 24 hours.

In another aspect, methods of making a medical device are provided. In one embodiment of a method, a male luer lock fitting with a conical tip having an outside surface that mates with a female luer lock fitting of another medical device includes roughening at least one of the outside surface of the conical tip and the surface of the female luer lock fitting so that after assembly of the male luer lock fitting and the female luer lock fitting while liquid is present between the male conical tip and female luer lock fitting, the removal torque after the fittings have been assembled for 24 hours is less than the removal torque of a conventional male luer lock fitting from a female luer lock fitting assembled under similar conditions for 24 hours.

Another aspect pertains to methods of assembling a medical device. In one or more embodiments, a method of assembling a medical device comprises using a twisting motion to connect a male luer lock fitting of a first medical device having a conical tip with an outside surface to a female luer lock fitting of another medical device while liquid is present between the male conical tip and female luer lock fitting. Either one or both of the outside surfaces of the male conical tip or a surface of the female luer lock fitting is roughened so that after the fittings have been assembled for 24 hours, the disassembly torque is less than that of a conventional male luer lock fitting connected to a female luer lock fitting under similar conditions and assembled for 24 hours.

### BRIEF DESCRIPTION OF THE DRAWING

So that the manner in which the above recited features of the present invention can be understood in detail, a more particular description of the invention, briefly summarized above, may be had by reference to embodiments, some of which are illustrated in the appended drawing. It is to be noted, however, that the appended drawing illustrates a typical embodiment of this invention and are therefore not to be considered limiting of the scope, for the invention may admit to other equally effective embodiments.

FIG. 1 schematically illustrates the components of a medical device with a male luer lock fitting connected to a medical device having a female luer lock fitting.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

As used herein, “luer lock” used in conjunction with luer lock connectors and luer lock fittings means a connector that is disassembled primarily by the application of torque as described in ISO 594-2, as distinguished from luer fittings that are disassembled primarily by the application of axial force, as described in ISO 594/1.

It has been determined that when a male luer lock fitting with a conical tip and a female luer lock fitting are assembled in the presence of liquid with sufficient torque, and if allowed to remain connected for extended periods of time, such conditions can require excessive torque to disassemble the assembled components, causing the male conical tip to fail and break off at the base. It has been determined that by adjusting the surface finish on at least the male conical tip of a luer lock connection of a medical device, breakage during disassembly can be eliminated.

While the present application should not be bound by a particular theory, it is believed that when a male luer lock fitting and female luer lock fitting are assembled under wet conditions, high interface forces between the fittings are created due to the reduced friction between components. Over time, the conditions that led to reduced friction during assembly change, resulting in an increased state of friction during disassembly. During removal, the torque required to overcome the interference force and friction between the fittings can cause the male conical tip to fail and break. It is believed that by affecting the mode of lubrication during assembly, friction during assembly can be significantly increased. Increasing friction during assembly consequently reduces the potential for high interference forces, resulting in lower removal torques during disassembly. Several variables contribute to the extent to which such male conical tips may fail and break. These include the specific fluid present at the interface between the components, the extent of the wetness of the interface, the time between assembly and disassembly, the magnitude of the assembly torque, the materials of composition of both the male luer lock fitting and the female luer lock fitting, and the surface finishes of both the male luer lock fitting and the female luer lock fitting.

Quite unlike the situation encountered with luer connectors that are assembled and disassembled by the application of primarily axial force (as described in International Standard ISO 594/1), the presence of fluid between the male and female fittings of luer lock connectors assembled by an application torque or twisting motion can require increased torque to disassemble the fittings. Under certain conditions such as those discussed above, the increased torque can cause failure and breakage of the conical tip of the male luer lock fitting. On the other hand, as is known in the art and described in the literature, luer connectors that do not include a threaded collar and are assembled by the application of primarily axial force can experience problems with accidental disengagement of the fittings, because the fluid causes a reduced coefficient of friction between the male and female fittings. Thus, attempts have been made for these types of fittings described in ISO 594/1, which are assembled and disassembled primarily by the application of axial force, to increase the coefficient of friction between the fittings when they are wet to require a greater axial force to disassemble the fittings. Thus, the literature has taught that roughening the surface of the male or female fitting will increase the axial force to disassemble male and female luer fittings having fluid present between the fittings. Therefore, the provision of a roughened surface on a male luer lock fitting assembled to a female luer lock fitting by application of torque would not be expected to decrease the torque required to disassemble the fittings.

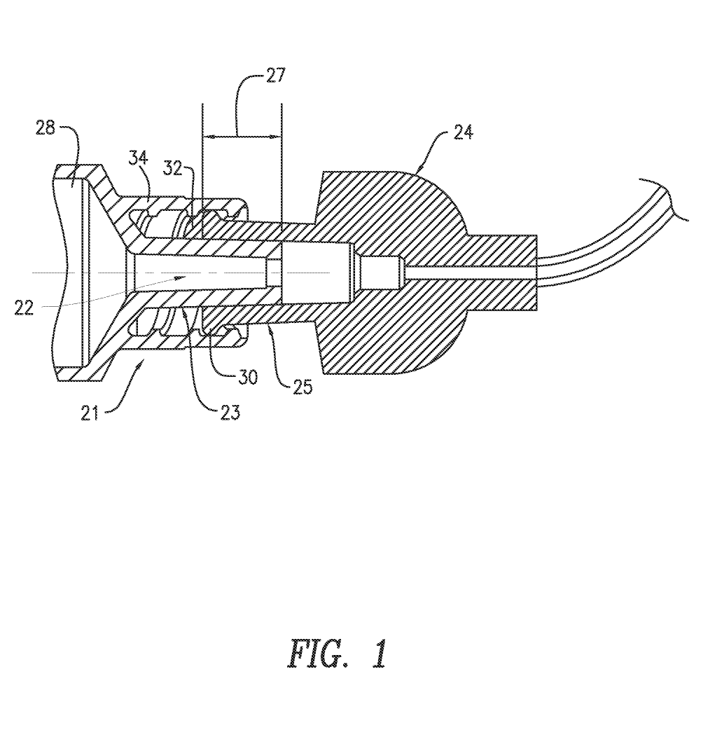

FIG. 1 shows a schematic representation of a connection between a syringe 21 having a male luer lock fitting 22 and a medical device 24 having a tubular female luer lock fitting 25, similar to the device shown and described in ISO 594-2. In the non-limiting embodiment shown, the male luer lock fitting 22 is shown as being disposed on a syringe tip 28. It will be appreciated that the luer lock fitting could be disposed on any type of medical device. The female luer lock fitting 25 includes radially extending lugs 30 that are adapted to engage screw threads 32 provided on internally threaded collar 34 arranged coaxially with male luer lock fitting 22. The threaded collar 34 shown in FIG. 1 is shown as permanently connected to the syringe, but it will be appreciated that collar 34 could be a rotational internally threaded collar. The male luer lock fitting 22 is inserted into the female luer lock fitting 25. The male luer lock fitting 22 is provided with a conical tip having a rough outer surface 23 to increase the coefficient of friction during assembly of the male luer lock fitting 22 and female luer lock fitting 25 in the presence of liquid. Assembly of the fittings occurs when an appropriate amount of torque is applied to the female luer lock fitting 25 so that lugs 30 engage the screw threads 32 of the threaded collar 34 to advance the female luer lock fitting 25 onto the male luer lock fitting 22 to a final assembly depth 27. As shown, the assembly depth 27 is the distance for which there is contact between surfaces of the male luer lock fitting 22 and the female luer lock fitting 25. Disassembly of the male luer lock fitting 22 and female luer lock fitting 25 is achieved by applying an unscrewing torque to the female luer lock fitting 25. ISO 594-2 states that while the unscrewing torque is being applied, the practitioner should not apply any axial force or inertial loading during removal.

The invention will be further described by reference to examples.

### EXAMPLES

The examples described are not to be taken as limiting the scope of the invention. Samples were prepared using different surface roughening techniques. A collection of these samples and the average surface roughness is presented in Table 1. It should be noted that the scope of the invention is not limited to the surface roughening techniques listed in Table 1.

TABLE 1
Average Surface Roughness from Various Manufacturing Processes

| | Average Measured |
|---|---|
| Sample Core | Roughness (μ-in) |
| SPI A-2 Finish | 6.2 |
| 600 Cross Hatch | 6.7 |
| 150 Grit Vapor Hone | 12.4 |
| EDM Charmilles 12 | 21.2 |
| EDM Charmilles 16 | 42.3 |
| EDM Charmilles 21 | 64.6 |

The 600 cross-hatch finish is a finish that is on 60 ml Luer Lok® products sold by the assignee of the present invention. The finish is provided by injection molding the product in a mold that has been finished using 600 grit sandpaper on the surface of the mold forming the tapered conical tip. The 150 grit Vapor Hone sample was created using a media blast using 150 grit media on the surface of the mold forming the conical tip. EDM refers to Electrodischarge Machining of the surface of the mold forming the conical tip. The average surface roughness measurements in Table 1 were taken using a non-contact Veeco profilometer. The SPI A-2 Finish and 600 Cross Hatch finish samples are included as reference samples for conventional male luer lock fittings sold by the assignee of the present invention (a non-limiting example, 60 ml luer-lok syringes, BD Catalog Number 309653). Therefore, the average surface roughness measured for these samples is representative of the average surface roughness of conventional products.

Samples of a male luer lock fittings made from a semi-rigid plastic, polypropylene, with various surface roughening techniques were assembled with a female luer fitting made from a semi-rigid plastic, polyethylene terephthalate, in the presence of a liquid, meaning that liquid was covering at least a portion of the male luer lock fitting, so that liquid is present between the male luer lock fitting and female luer lock fitting during assembly. The liquid used was Bacteriostatic Water for Injection (BWFI), a 0.9% (9 mg/ml) solution of benzyl alcohol in water. The male luer lock fitting was assembled with the female luer lock fitting using a twisting motion and 45 in-oz of torque. The samples were allowed to sit for 24 hours before disassembling. The magnitude of torque required to disassemble the male conical fitting from the female luer fitting was measured along with an attribute assessment of whether or not the male conical tip failed during disassembly. In cases of tip failure, the disassembly torque reported was the torque to failure. The results of these studies are shown in Table 2. The skilled artisan will appreciate that various diluents may be used in clinical settings, therefore, various diluents were tested, and of which BWFI samples were observed in the testing to be a worst case scenario for removal torque in environments using fluids containing diluents.

TABLE 2
Disassembly Torque with BWFI as a Function of Surface Finish

| | | | | | | Standard | | |
|---|---|---|---|---|---|---|---|---|
| | | | | % | Mean | Deviation | Minimum | Maximum |
| Finish | Count | Failed | Passed | Failure | (in-lbs) | (in-lbs) | (in-lbs) | (in-lbs) |
| SPI A-2 Finish | 40 | 36 | 4 | 90% | 4.49 | 0.22 | 3.66 | 4.84 |
| 600 Cross Hatch | 40 | 32 | 8 | 80% | 4.52 | 0.14 | 4.00 | 4.74 |
| 150 Grit Vapor Hone | 40 | 0 | 40 | 0% | 3.18 | 0.50 | 2.36 | 4.38 |
| EDM Charmilles 12 | 40 | 0 | 40 | 0% | 2.19 | 0.63 | 1.48 | 3.84 |
| EDM Charmilles 18 | 40 | 0 | 40 | 0% | 2.53 | 0.28 | 1.76 | 2.88 |
| EDM Charmilles 21 | 40 | 0 | 40 | 0% | 2.36 | 0.32 | 1.84 | 2.94 |

Every sample exhibiting a disassembly torque greater than about 4.6 in-lbs failed during disassembly. Every sample exhibiting a disassembly torque less than about 4 in-lbs remained intact during disassembly. A mixture of intact and failing samples occurred at removal torques between these values. While Table 1 presents a limited sample of techniques to roughen the surface of the male conical tip, there are a large number of techniques which can be used to roughen the surface of the male luer lock tip. The techniques chosen for these examples are merely illustrative and the scope of the invention should not be limited to the finishes listed. In the Table above, the samples prepared by EDM achieved the best results and resulted in a lay particulate or protuberant finish. Any technique which results in a lay particulate surface can be utilized, but such a finish is not required. In addition, the present invention is not limited to roughening the surface of the male luer lock tip. In one or more embodiments, the inner surface of the female luer lock fitting in contact with the male conical tip during engagement can be roughened. In certain embodiments, both the male conical tip and female luer lock fitting can be roughened. The EDM samples had a lay particulate finish.

One or more embodiments of the invention provide a medical device with a luer lock fitting exhibiting a disassembly torque of less than about 4 in-lbs to separate the male luer lock conical fitting from a female luer lock fitting. In specific embodiments, the removal torque is less than about 3 in-lbs, and in a more specific embodiment, the removal torque is less than about 2.5 in-lbs.

Various embodiments of the invention use surface roughening techniques including, but not limited to, Electrodischarge Machining (EDM), vapor honing, cross hatching, polishing and combinations thereof to achieve a roughened surface on a luer lock fitting. However, the present invention should not be limited to a particular technique of achieving a surface finish, and other techniques including but not limited to chemical and electrochemical surface finishing techniques can be used. According to some embodiments of the invention, Electrodischarge Machining (EDM) is used to create the surface roughness on a mold, which then imparts through the molding process surface roughness on the outside surface of a male conical tip of a luer lock fitting. In one embodiment, the Charmilles level for the surface roughness is generally greater than about 12. In a specific embodiment, the Charmilles level is greater than about 18, and more specifically, greater than about 21. Other embodiments of the invention have the surface roughness created by vapor honing to about a 150 grit equivalent or lower.

Additional embodiments of the invention are directed toward methods of making a medical device comprising manufacturing a male luer lock fitting with a conical tip having an outside surface that mates with a female luer lock fitting of another medical device. The outside surface of the male conical tip is roughened so that after assembly of the male luer lock fitting and a female luer lock fitting in the presence of a liquid, the removal torque 24 hours after assembly is less than the removal torque of a conventional male luer lock fitting from a female luer lock fitting assembled for 24 hours. In other embodiments, the portion of the female luer lock fitting in contact with the conical tip is roughened to reduce the disassembly torque.

The disassembly torque of some embodiments, when the male conical fitting is assembled with the female luer lock fitting, in the presence of BWFI, is less than about 4 in-lbs. In specific embodiments, the removal torque is less than about 3 in-lbs, and in more specific embodiments, the removal torque is less than about 2.5 in-lbs.

Embodiments of the invention have the outside of the male conical fitting roughened using a technique selected from the group consisting of Electrodischarge Machining, vapor honing, cross hatching, polishing and combinations thereof to achieve the desired finish on the surface of a mold used to form the fitting. In specific embodiments, it is desirable that the surface finish is lay particulate or protuberant. Such surface finish can be provided by Electrodischarge Machining.

Further embodiments of the invention are directed to methods of assembling a medical device. In one or more embodiments, a method comprises connecting a male luer lock fitting of a medical device having a conical tip with an outside surface to a female luer lock fitting. The connection is made primarily with a twisting motion and in the presence of a liquid between the conical tip and female luer lock fitting. In one or more embodiments, the outside surface of the male conical tip is roughened, so that after being assembled for 24 hours, the disassembly torque is less than that of a conventional male luer lock fitting connected to a female luer lock fitting. As used herein, a conventional male luer lock fitting refers to a fitting with a 600 cross hatch finish or SPI A-2 finish.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A medical device comprising an adapter portion for connection to a fitting, the adapter portion being a male luer lock fitting having an internally threaded collar in a coaxial relationship with a male conical tip having an outside surface that mates upon application of torque with a female luer lock fitting having lugs that engage the threaded collar, one of the outside surface of the male conical tip and a surface of the female luer lock fitting being roughened such that after assembly of the male luer lock fitting with the female luer lock fitting when a liquid is present between the conical tip and female luer lock fitting, the torque required to remove the male luer lock fitting from the female luer fitting after being assembled for 24 hours is less than the torque required to remove a conventional male luer lock fitting from a female luer lock fitting after being assembled under similar conditions after being assembled for 24 hours.

2. The medical device of claim 1, wherein the torque required to remove the male luer lock fitting from the female luer lock fitting is less than about 4 in-lbs when the liquid is Bacteriostatic Water For Injection (BWFI) and each fitting is made from a semi-rigid plastic.

3. The medical device of claim 1, wherein the torque required to remove the male luer lock fitting from the female luer lock fitting is less than about 3 in-lbs when the liquid is BWFI and each fitting is made from a semi-rigid plastic.

4. The medical device of claim 1, wherein the torque required to remove the male luer lock fitting from the female luer lock fitting is less than about 2.5 in-lbs when the liquid is BWFI and each fitting is made from a semi-rigid plastic.

5. The medical device of claim 1 wherein the surface of the male conical tip is roughened using a mold to form the tip that has been roughed using a technique selected from Electrodischarge Machining (EDM), vapor honing, cross hatching, polishing and combinations thereof.

6. The medical device of claim 5 wherein the EDM is at a Charmilles level greater than about 12.

7. The medical device of claim 5 wherein the EDM is at a Charmilles level greater than about 18.

8. The medical device of claim 5 wherein the EDM is at a Charmilles level greater than about 21.

9. The medical device of claim 5 wherein the surface finish on the male conical tip is a lay particulate finish.

10. The medical device of claim 5 wherein the vapor honing creates a surface equivalent to or less than 150 grit.

11. A method of making a medical device comprising:
manufacturing a male luer lock fitting with a conical tip having an outside surface that mates with a female luer lock fitting of another medical device; roughening the outside surface of one of the male conical tip and a surface of the female luer lock fitting so that after assembly of the male luer lock fitting and the female luer lock fitting with liquid present between the male conical tip and female luer lock fitting, the removal torque 24 hours after assembly is less than the removal torque of a conventional male luer lock fitting from a female luer lock fitting 24 hours after assembly.

12. The method of claim 11 wherein the outside surface of the male luer lock fitting is roughened so that the removal torque is less than about 4 in-lbs when the liquid is Bacteriostatic Water For Injection (BWFI) and each fitting is made from a semi-rigid plastic.

13. The method of claim 11 wherein the outside surface of the male luer lock fitting is roughened so that the removal torque is less than about 3 in-lbs when liquid is BWFI and each fitting is made from a semi-rigid plastic.

14. The method of claim 11 wherein the outside surface of the male luer lock fitting is roughened so that the removal torque is less than about 2.5 in-lbs when the liquid is BWFI and each fitting is made from a semi-rigid plastic.

15. The method of claim 11 wherein roughening the outside surface of the male conical tip or a surface of the female luer lock fitting includes roughening the surface of a the surface of a mold that is used to form the tip using a technique selected from Electrodischarge Machining, vapor honing, cross hatching, polishing and combinations thereof.

16. A method of assembling a medical device comprising
using twisting motion to connect a male luer lock fitting of a first medical device having a conical tip with an outside surface to a female luer lock fitting while liquid is present between the conical tip and the female luer lock fitting, the outside surface of the male conical tip or a surface of the female luer lock being roughened so that 24 hours after assembly, the disassembly torque is less than the disassembly torque of a conventional male luer lock fitting connected to a female luer lock fitting 24 hours after assembly under similar conditions.

17. The method of claim 16 wherein the disassembly torque in the presence of bacteriostatic water for injection (BWFI) is less than about 0.4 in-lbs and each fitting is made from a semi-rigid plastic.

18. The method of claim 16 wherein the disassembly torque when the liquid is BWFI is less than about 0.3 in-lbs and each fitting is made from a semi-rigid plastic.

19. The method of claim 16 wherein the disassembly torque when the liquid is BWFI is less than about 0.25 in-lbs and each fitting is made from a semi-rigid plastic.
